(19) United States
(12) Patent Application Publication
(10) Pub. No.: US 2017/0246175 A1
(43) Pub. Date: Aug. 31, 2017

(54) SUSTAINED TIMOLOL MALEATE DELIVERY FROM LIPOSOMES FOR GLAUCOMA THERAPY AND OCCULAR HYPERTENSION

(71) Applicant: Nanyang Technological University, Singapore (SG)

(72) Inventors: Subramanian Venkatraman, Singapore (SG); Yin Chiang Freddy Boey, Singapore (SG); Jayaganesh V. Natarajan, Singapore (SG)

(21) Appl. No.: 15/513,010

(22) PCT Filed: Sep. 23, 2015
(86) PCT No.: PCT/SG2015/050337

§ 371 Date: Mar. 21, 2017

Related U.S. Application Data
(60) Provisional application No. 62/054,419, filed on Sep. 24, 2014.

(51) Int. Cl. A61K 31/5377; A61K 31/728; A61K 9/08; A61K 9/00; A61K 9/127; A61K 31/5575

(52) U.S. Cl. CPC A61K 31/5377; A61K 9/1272; A61K 31/5575; A61K 9/08; A61K 9/0048; A61K 31/728; A61K 9/0019; A61K 9/1277

(57) ABSTRACT

Various embodiments relate to the field of liposomal formulations for drug delivery, in particular, liposomal formulations for ocular drug delivery. More specifically, various embodiments relate to sustained timolol maleate delivery from liposomes for glaucoma therapy and ocular hypertension.

### CROSS-REFERENCE TO RELATED APPLICATION

This application claims the benefit of priority of United States of America Provisional Patent Application No. 62/054,419, filed Sep. 24, 2014, the contents of which being hereby incorporated by reference in its entirety for all purposes.

### TECHNICAL FIELD

Various embodiments relate to the field of liposomal formulations for drug delivery, in particular, liposomal formulations for ocular drug delivery. More specifically, various embodiments relate to sustained timolol maleate delivery from liposomes for glaucoma therapy and ocular hypertension.

### BACKGROUND

The pressure within the eyeball is naturally maintained by a continuous flow of aqueous humour produced by ciliary body. It drains out the excess eyeball fluid through channels called the trabecular meshwork. If the outflow is blocked, the aqueous humour builds up inside the eye, increasing the pressure within the eyeball. This pressure needs to be reduced, as otherwise it can damage the optic nerve, resulting in an optic neuropathy and irreversibly impair vision as a result. This condition is known as glaucoma—a disease that affects more than 60 million worldwide and is the second leading cause of blindness. Intraocular pressure (IOP) remains the key modifiable risk factor in glaucoma. Current treatment strategies include the use of eye drops; but this often leads to variable therapeutic outcomes, side effects due to prolonged administration and patient non-compliance.

Therefore, there remains an unmet need for an effective and alternative treatment strategy for glaucoma therapy and ocular hypertension.

### SUMMARY

It is herein disclosed sustained release formulations of timolol maleate loaded into liposomal nanocarriers that offer an alternative and effective treatment strategy for glaucoma therapy and ocular hypertension. Present approach involves administering the liposomal nanocarriers via a subconjunctival route (a safer alternative route in comparison to intravitreal route) and maintaining the IOP lowering at least for five-six months with a single injection.

Present studies demonstrate that high entrapped drug concentration of timolol maleate (up to 1.5 mg/ml) loaded into the core of the liposomes using an ammonium sulphate gradient has been achieved. In addition, controlled release of timolol maleate from the liposomal nanocarriers beyond three weeks in an in vitro dialysis set up has been achieved, and the release was found to be dependent on the concentration of the additive cholesterol, with the slowest release observed for liposomes containing 40 mol % of cholesterol.

Accordingly, various embodiments relate to a liposomal formulation for ocular drug delivery, comprising:
liposomes each comprising a core surrounded by one or more lipid bilayers, and timolol maleate comprised in the core of each liposome,
(a) wherein the one or more lipid bilayers are comprised of a mixture of 50-80 mol % of a lipid and 20-50 mol % of a steroid alcohol, wherein the lipid is comprised of glyceride, phosphatidylcholine, and/or sphingolipid; or
(b) wherein the one or more lipid bilayers are comprised of a mixture of 50-80 mol % of a neutral lipid and 20-50 mol % of a charged lipid, wherein the neutral lipid and the charged lipid are comprised of glyceride, phosphatidylcholine, and/or sphingolipid.

Various embodiments further relate to a pharmaceutical composition comprising a pharmaceutically acceptable carrier and a liposomal formulation mentioned earlier.

Various embodiments further relate to a use of a liposomal formulation or a pharmaceutical composition mentioned earlier in the treatment or prevention of glaucoma or ocular hypertension, the use comprising administering the liposomal formulation or the pharmaceutical composition to a subject in need thereof.

Various embodiments further relate to a method of providing a sustained release of timolol maleate of minimum 10 days, comprising administering a liposomal formulation or a pharmaceutical composition mentioned earlier by subconjunctival injection.

Various embodiments further relate to a method of preparing a liposomal formulation comprising liposomes each comprising a core surrounded by one or more lipid bilayers, and timolol maleate comprised in the core of each liposome, wherein the one or more lipid bilayers are comprised of a mixture of 50-80 mol % of a lipid and 20-50 mol % of a steroid alcohol, wherein the lipid is comprised of glyceride, phosphatidylcholine, and/or sphingolipid, the method comprising:
mixing the lipid with the steroid alcohol in an organic phase;
evaporating the organic phase mixture to obtain a thin film of the lipid and steroid alcohol;
hydrating the thin film with ammonium sulfate solution to form vesicles;
suspending the vesicles in a salt solution;
adding a drug solution to the suspension; and
incubating the suspension containing the vesicles and drug solution.

Various embodiments further relate to a method of preparing a liposomal formulation comprising liposomes each comprising a core surrounded by one or more lipid bilayers, and timolol maleate comprised in the core of each liposome, wherein the one or more lipid bilayers are comprised of a mixture of 50-80 mol % of a neutral lipid and 20-50 mol % of a charged lipid, wherein the neutral lipid and the charged lipid are comprised of glyceride, phosphatidylcholine, and/or sphingolipid, the method comprising:
mixing the neutral lipid and the charged lipid in an organic phase;
evaporating the organic phase mixture to obtain a thin film;
hydrating the thin film with ammonium sulfate solution to form vesicles;
suspending the vesicles in a salt solution;
adding a drug solution to the suspension; and
incubating the suspension containing the vesicles and drug solution.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 shows the percentage cumulative average release versus days of liposome P1 according to the examples.

FIG. 2 shows the amount of drug released from 1 ml of liposome suspension of P1 according to the examples.

FIG. 3 shows the percentage cumulative average release versus days of liposome P2 according to the examples.

FIG. 4 shows the amount of drug released from 1 ml of liposome suspension of P2 according to the examples.

FIG. 5 shows the percentage cumulative average release versus days of liposome P3 according to the examples.

FIG. 6 shows the amount of drug released from 1 ml of liposome suspension of P3 according to the examples.

FIG. 7 shows a comparison of cumulative average release of timolol maleate from plain, DPPC/Cholesterol (80:20 mol %), and DPPC/Cholesterol (60:40 mol %) liposomes according to the examples.

FIG. 8 shows the in vitro drug release profiles from plain, DPPC/DPPG (80:20 mol %), and DPPC/DOTAP (80:20 mol %) liposomes according to the examples.

FIG. 9 shows the cumulative release of timolol maleate from sphingomyelin liposomes containing 40 mol % cholesterol according to the examples.

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural and chemical changes may be made without departing from the scope of the invention. The various embodiments are not necessarily mutually exclusive, as some embodiments can be combined with one or more other embodiments to form new embodiments.

Various embodiments provide liposomal formulations for ocular drug delivery, that is, drug delivery to an eye. The liposomal formulations exhibit improved sustained release of the drugs from the liposomal formulations. Additionally, such liposomal formulations also circumvent the need of patient compliance under strict regime. For example, administering subconjunctival injections of sustained release of the drug loaded in liposomes exhibit excellent effectiveness of delivering superior therapeutic concentration of drug to the eye without the need to depend on patient compliance.

Accordingly, various embodiments relate to a liposomal formulation for ocular drug delivery, comprising:
liposomes each comprising a core surrounded by one or more lipid bilayers, and timolol maleate comprised in the core of each liposome,
   (a) wherein the one or more lipid bilayers are comprised of a mixture of 50-80 mol % of a lipid and 20-50 mol % of a steroid alcohol, wherein the lipid is comprised of glyceride, phosphatidylcholine, and/or sphingolipid; or
   (b) wherein the one or more lipid bilayers are comprised of a mixture of 50-80 mol % of a neutral lipid and 20-50 mol % of a charged lipid, wherein the neutral lipid and the charged lipid are comprised of glyceride, phosphatidylcholine, and/or sphingolipid.

In present context, the term “liposomal formulation” refers to a formulation of liposomes, wherein liposomes are artificially prepared vesicles made of lipid bilayer, which is defined as a thin membrane made of two layers of lipid molecules. Lipid bilayer may be in a form of a single or one lipid bilayer, or of multiple lipid bilayers. Liposomes may be filled or loaded with drugs, and used to deliver drugs for cancers and other diseases. The drugs may therefore be associated in the liposomes, wherein the term “associated”, “load”, “incorporated”, or “encapsulated” as used interchangeably, may generally refer to being coupled, connected, related, linked or encapsulated.

As mentioned above, the liposomes comprise glyceride, phosphatidylcholine, and/or sphingolipid. In various embodiments, liposomes that are made up of phospholipid bilayers surrounding an aqueous core may preferably contain a drug, such as but is not limited to timolol maleate. It is usually of 10 nm to 10 μm or greater in diameter. They are classified as unilamellar vesicles and multilamellar vesicles (MLVs). The unilamellar vesicles are further classified into small unilamellar vesicles (SUVs), large unilamellar vesicles (LUVs) and giant unilamellar vesicles (GUVs). Unilamellar vesicles are composed of single bilayer of phospholipids encapsulating aqueous core whereas the multilamellar vesicles are composed of multiple phospholipids bilayers. Liposomes can entrap both hydrophilic and lipophilic drugs by partitioning them into hydrophobic domains.

Straight forward hydration of lipids produces MLVs. The MLVs may pass through filters, for example, polycarbonate filters having a filter pore size of about 2 μm. The MLVs obtained may have a size distribution of about 0.9-1.5 μm.

Unilamellar vesicles may be produced directly from MLVs by extrusion or sonication or, alternatively, may be obtained by reverse phase or detergent removal procedures. By extrusion, for example extruding 5 times through 0.2 μm polycarbonate filters, 5 times through 0.1 μm polycarbonate filters, and 10 times through 0.08 μm polycarbonate filters sequentially, the MLVs may be downsized to LUVs with a mean size or diameter of about 100 nm, for example 100±20 nm.

Sonication may be typically used to obtain SUVs.

SUVs may also be obtained by extrusion through filters, for example polycarbonate filters, with smaller pore sizes as compared to the case for LUVs.

LUVs are suitable for ocular delivery because they are optically clear and do not increase in size upon storage. It is important for a liposomal formulation to be optically clear for ocular drug delivery, especially for sustained release of ocular drug encapsulated in the liposomal formation so that clear vision and sight of the eye can be maintained throughout the administration process and the drug release process.

In various embodiments, the liposomes are small unilamellar vesicles (SUV) or large unilamellar vesicles (LUV) or multilamellar vesicles. In one embodiment, the liposomes may have a mean diameter of less than 1 μm. In yet another embodiment, the liposomes may have a mean diameter of about 100 nm to about 300 nm. For example, the liposomes may have a mean diameter of about 20 nm to about 50 nm. In context of various embodiments, the term “mean diameter” may generally refer to a mathematical average of a set of diameters, each diameter being taken for each liposome in a liposome population. The term “about” associated with the measure of a diameter may generally refer to an approximate which may be due to the imperfect circular structure of a liposome that may be elliptical in shape.

The liposomes may be made up of various grades of phospholipids and some other ingredients. Such other ingredients may include a steroid alcohol, such as phytosterol, zoosterol, or a mixture thereof. In various embodiments, the steroid alcohol has a general formula

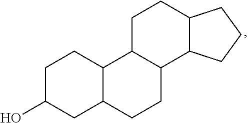

wherein any of the carbon atom is optionally substituted, preferably with a C1-C10 alkyl.

The term “alkyl”, alone or in combination, refers to a fully saturated aliphatic hydrocarbon. In certain embodiments, alkyls are optionally substituted. In certain embodiments, an alkyl comprises 1 to 10 carbon atoms, wherein a numerical range, such as “1 to 10” or “C1-C10”, refers to each integer in the given range, e.g. “C1-C10 alkyl” means that an alkyl group comprising only 1 carbon atom, 2 carbon atoms, 3 carbon atoms, etc., up to and including 10 carbon atoms. Examples of alkyl groups include, but are not limited to, methyl, ethyl, n-propyl, isopropyl, n-butyl, isobutyl, sec-butyl, tert-butyl, tert-amyl, pentyl, hexyl, heptyl, octyl and the like.

Preferably, the steroid alcohol has the following structure

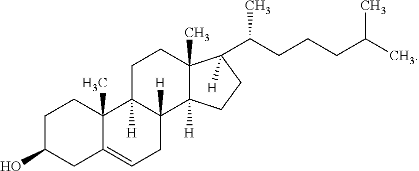

In preferred embodiments where the steroid alcohol comprises cholesterol, the cholesterol acts as a vesicle stabilizer by improving the rigidity of the bilayer membrane. The cholesterol may also include derivatives thereof, for example, cholestanol, dihydrocholesterol, cholesteryl esters, phytosterol, sitosterol, stigmasterol and campesterol.

Various amounts of steroid alcohol or cholesterol may be comprised in the liposome. For example, the cholesterol may be of an amount of about 20 mol % to about 50 mol % based on the total amount of the lipid and cholesterol, such as about 20 mol % to about 40 mol %, 30 mol % to about 50 mol %, or about 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, or 50 mol %.

In one embodiment, the cholesterol may be of an amount of about 40 mol %.

Vesicle morphology liposome depends on the various properties like surface charge, size, surface hydration and fluidity of lipid bilayers. The use of charge inducers is not unknown. The cornea generally carries negative charge and hence the positively charged liposomes display better corneal permeation. Moreover, the charged liposomes may exhibit less aggregation tendency as compared to neutral liposomes.

In the context of various embodiments, the term “phosphatidylcholine” may generally refer to a class of phospholipids (amphipathic lipids) that incorporate choline as a headgroup with one or more phosphate groups attached to it, and more specifically, refer to a lipid consisting of a glycerol bound to two fatty acids and a phosphate group. The term “lipid” may generally refer to an oily organic compound insoluble in water but soluble in organic solvents.

For example, the phosphatidylcholines may be selected from the group consisting of 1,2-dioleoyl-sn-glycero-3-phosphocholine (DOPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1-palmitoyl-2-oleoyl-sn-glycero-3-phosphocholine (POPC), egg phosphatidylcholine (egg PC), soy phosphatidylcholine (soy PC), hydrogenated phosphatidylcholine (HPC), and a mixture thereof.

In one embodiment, the phosphatidylcholine may be 1,2-dipalmitoyl-sn-glycero-3-phosphocholines (DPPC) having the structure

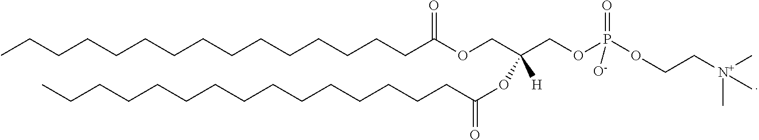 [SMILES: CCCCCCCCCCCCCCCC(=O)OC[C@H](COP(=O)([O-])OCC[N+](C)(C)C)OC(=O)CCCCCCCCCCCCCCC]

In one embodiment, the structure of egg PC is

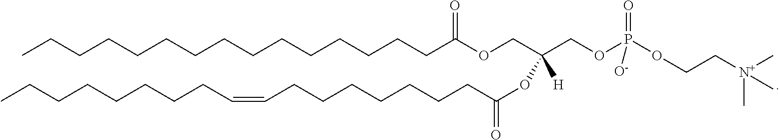 [SMILES: CCCCCCCC/C=C\CCCCCCCC(=O)O[C@H](COC(=O)CCCCCCCCCCCCCCC)COP(=O)([O-])OCC[N+](C)(C)C]

In one embodiment, the structure of soy PC is

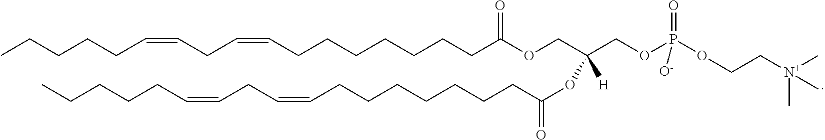 [SMILES: CCCCC/C=C\C/C=C\CCCCCCCC(=O)OC[C@H](COP(=O)([O-])OCC[N+](C)(C)C)OC(=O)CCCCCCC/C=C\C/C=C\CCCCC]

In various embodiments, the sphingolipid may comprise at least one unsaturated fatty acid moiety. For example, the sphingolipids may comprise hexadecanoylsphingomyelin or Egg sphingomyelin having the structure

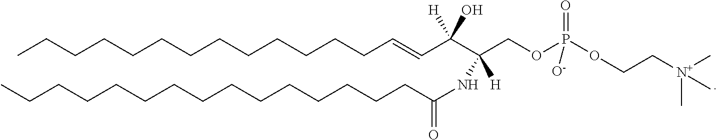 [SMILES: CCCCCCCCCCCCC/C=C/[C@@H](O)[C@H](COP(=O)([O-])OCC[N+](C)(C)C)NC(=O)CCCCCCCCCCCCCCC]

In various embodiments, timolol maleate is comprised in the core and another ocular drug is comprised in the lipid bilayer of each liposome. In exemplary embodiments, the liposomal formulation may further comprise:
additional liposomes each comprising a core surrounded by one or more lipid bilayers; and
another ocular drug different from timolol maleate comprised in the one or more lipid bilayers of each additional liposome.

The another ocular drug may be selected from the group consisting of latanoprost, bimatoprost, travoprost, carboprosttrometamol, gemeprost, sulprostone, dinoprostone (PGE2), alprostadil (PGE1), beroprost, iloprost, epoprostenol, treprostinil, misoprostol, enoprostil, omoprostil, limaprost. unoprostone isopropyl, arthrotec, and a mixture thereof. In one embodiment, the another ocular drug is latanoprost.

The timolol maleate to lipid mole ratio in the liposomal formulation is generally not limited to any particular value. Nevertheless, in preferred embodiments the liposomal formulation comprises a timolol maleate to lipid mole ratio of about 0.01 to about 0.30, more preferably 0.01, 0.05, 0.10, 0.15, 0.20, 0.25, or 0.30.

In various embodiments, the lipid bilayer of the liposomal formulation is comprised of a neutral lipid and a negatively charged lipid.

The neutral lipid may comprise 1,2-dipalmitoyl-sn-glycero-3-phosphocholine (DPPC), 1,2-dioleoyl-sn-glycero-3-phosphocholines (DOPC), 1,2-Dilauroyl-sn-glycero-3-phosphocholines (DLPC), 1,2-Dimyristoyl-sn-glycero-3-phosphocholines (DMPC), 1,2-Distearoyl-sn-glycero-3-phosphocholines (DSPC), L-α-phosphatidylcholine or 95% Egg phosphatidylcholines (eggPC 95%), 1-palmitoyl-2-oleoylphosphatidylcholine (POPC), L-α-phosphatidylcholine, hydrogenated (Soy) or mixtures thereof.

The negatively charged lipid may comprise 1,2-dipalmitoyl-sn-glycero-3-phospho-(1′-rac-glycerol) (sodium salt) (DPPG), (L-α-phosphatidylglycerol (Egg, Chicken) (sodium salt) (EggPG), L-α-phosphatidylglycerol (Soy) (sodium salt) (Soy PG), 1,2-dimyristoyl-sn-glycero-3-phospho-(1′-rac-glycerol) (sodium salt) (DMPG), 1,2-dilauroyl-sn-glycero-3-phospho-(1′-rac-glycerol) (sodium salt) (DLPG), 1,2-distearoyl-sn-glycero-3-phospho-(1′-rac-glycerol) (sodium salt), L-α-phosphatidic acid (Egg, Chicken) (sodium salt) (EggPA), L-α-phosphatidic acid (Soy) (sodium salt) (SoyPA), 1,2-dilauroyl-sn-glycero-3-phosphate (sodium salt) (DLPA), 1,2-dimyristoyl-sn-glycero-3-phosphate (sodium salt) (DMPA), 1,2-dipalmitoyl-sn-glycero-3-phosphate (sodium salt) (DPPA), 1,2-distearoyl-sn-glycero-3-phosphate (sodium salt) (DSPA) and mixtures thereof.

In one preferred embodiment where the lipid bilayer of the liposomal formulation comprises a neutral lipid and a negatively charged lipid, the lipid bilayer comprises 80 mol % of DPPC and 20 mol % DPPG.

In alternative embodiments, the lipid bilayer of the liposomal formulation is comprised of a neutral lipid and a positively charged lipid.

As described earlier, the neutral lipid may comprise 1,2-dipalmitoyl-sn-glycero-3-phosphocholine (DPPC), (1,2-dioleoyl-sn-glycero-3-phosphocholines (DOPC), 1,2-Dilauroyl-sn-glycero-3-phosphocholines (DLPC), 1,2-Dimyristoyl-sn-glycero-3-phosphocholines (DMPC), 1,2-Distearoyl-sn-glycero-3-phosphocholines (DSPC), L-α-phosphatidylcholine or 95% Egg phosphatidylcholines (eggPC 95%), 1-palmitoyl-2-oleoylphosphatidylcholine (POPC), L-α-phosphatidylcholine, hydrogenated (Soy) (HSPC) and mixtures thereof.

The positively charged lipid may comprise 1,2-dioleoyl-3-trimethylammonium-propane (DOTAP), (1,2-dioleoyl-3-trimethylammonium-propane (chloride salt) (DOTAP), 1,2-dimyristoyl-3-trimethylammonium-propane (chloride salt) (DMTAP), 1,2-dipalmitoyl-3-trimethylammonium-propane (chloride salt) (DPTAP), 1,2-stearoyl-3-trimethylammonium-propane (chloride salt) (DSTAP), Dimethyldioctadecylammonium (Bromide Salt) (DDAB), 1,2-di-O-octadecenyl-3-trimethylammonium propane (chloride salt) (DOTMA) and mixtures thereof.

In one preferred embodiment where the lipid bilayer of the liposomal formulation comprises a neutral lipid and a positively charged lipid, wherein the lipid bilayer comprises 80 mol % of DPPC and 20 mol % DOTAP.

Various embodiments relate to a pharmaceutical composition comprising the liposomal formulation described earlier and a pharmaceutically acceptable carrier.

The pharmaceutical composition may be in a form of an ophthalmic solution. In various embodiments, the pharmaceutical composition is for use in ocular drug delivery in a form of an injection solution or a viscous aqueous vehicle. In another embodiment, the viscous aqueous vehicle comprises an aqueous solution of polysaccharides. The polysaccharides may be hyaluronic acid. By applying the liposomal formulation using the viscous aqueous vehicle, the liposomal formulation may retain in the eye for longer without clearance.

A method of producing the liposomal formulation may include thin-film hydration technique. Thin-film hydration technique enables uniform encapsulation of drug within the phospholipids of the liposomal formulation. In the context of various embodiments, the technique of thin-film hydration generally refers to a technique that is performed by firstly dissolving basic components forming a liposome membrane in an organic solvent such as chloroform, secondly subsequently subjecting the solution to a rotary evaporator to distill off the solvent by heating under reduced pressure to form a thin film on the inner side of the evaporator, and thirdly hydrating the thin film with a phosphate buffer solution, ammonium sulphate solution, or a HEPES-HBSS solution in a warm water bath. When the drug is water-soluble, it is dissolved in a solution for hydration, and when the drug is water-insoluble, it is dissolved in an organic solvent together with the liposome-forming components. In various embodiments, the method may further comprise downsizing the liposomal formulation by extrusion through a filter or by sonication.

Various embodiments relate to a method for treating or preventing glaucoma or ocular hypertension, comprising administering the liposomal formulation or the pharmaceutical composition to a subject in need thereof. The method comprises administering the liposomal formulation or pharmaceutical composition by subconjunctival injection to provide sustained release of timolol maleate. By applying the liposomal formulation via injection into the conjuctival sac, there may be provided a greater retention of the liposomal formulation in the eye, including a sustained release of timolol maleate of minimum 10 days.

Various embodiments provide for a method of preparing a liposomal formulation comprising liposomes each comprising a core surrounded by one or more lipid bilayers, and timolol maleate comprised in the core of each liposome, wherein the one or more lipid bilayers are comprised of a mixture of 50-80 mol % of a lipid and 20-50 mol % of a steroid alcohol, wherein the lipid is comprised of glyceride, phosphatidylcholine, and/or sphingolipid. The method comprises:
mixing the lipid with the steroid alcohol in an organic phase;
evaporating the organic phase mixture to obtain a thin film of the lipid and steroid alcohol;
hydrating the thin film with ammonium sulfate solution to form vesicles;
suspending the vesicles in a salt solution;
adding a drug solution to the suspension; and
incubating the suspension containing the vesicles and drug solution.

Various embodiments further provide for a method of preparing a liposomal formulation comprising liposomes each comprising a core surrounded by one or more lipid bilayers, and timolol maleate comprised in the core of each liposome, wherein the one or more lipid bilayers are comprised of a mixture of 50-80 mol % of a neutral lipid and 20-50 mol % of a charged lipid, wherein the neutral lipid and the charged lipid are comprised of glyceride, phosphatidylcholine, and/or sphingolipid. The method comprises:
mixing the neutral lipid and the charged lipid in an organic phase;
evaporating the organic phase mixture to obtain a thin film;
hydrating the thin film with ammonium sulfate solution to form vesicles;
suspending the vesicles in a salt solution;
adding a drug solution to the suspension; and
incubating the suspension containing the vesicles and drug solution.

In order that the invention may be readily understood and put into practical effect, particular embodiments will now be described by way of the following non-limiting examples.

### EXAMPLES

### Preparation of Timolol Maleate Liposomes

### Materials

Lipids, 1,2-dipalmitoyl-sn-glycero-3-phosphocholine (DPPC) and sphingomyelin, were purchased from NOF Corporation, Japan. Cholesterol (95%) was purchased from Sigma, USA. Polycarbonate (PC) filter membranes (sizes 0.2 μm, 0.1 μm) and drain discs were purchased from Northern Lipids Inc, Canada. Ammonium sulfate and sodium chloride were acquired from Sigma, USA, and used without additional purification. Cellulose ester dialysis bag (100,000 MWCO) was purchased from Fisher Scientific Pte Ltd, USA. The ultrapure water used in all washing steps and experiments was obtained from Millipore system with resistivity higher than 18.2 MΩ cm⁻¹.

### Preparation Strategy

### (1) Preparation of Drug-Free Preformed Liposomes

Firstly, lipid was placed in a vacuum desiccator for 1 hour to remove any residual moisture before weighing. Each batch of liposome was prepared in 5 ml per batch, with an initial lipid concentration of 18 millimolar (mM). Cholesterol was incorporated into the lipids in various mole %. DPPC pure (i.e. without cholesterol), DPPC/Cholesterol (80:20 mol %), DPPC/Cholesterol (60:40 mol %) and sphingomyelin/Cholesterol (60:40 mol %) were prepared. Lipids and cholesterol in fixed ratios were measured and taken in a round bottom flask and dissolved in an organic phase mixture that contained methanol and chloroform in a ratio of 1:2. Subsequently, the flask was rotated in a rotary operator and operated under reduced pressure for 1 hour maintained in a water bath temperature of 40° C. to remove the organic phase, ultimately leaving behind a thin film of lipids covering the bottom of the flask. To the thin lipid film, 5 ml of ammonium sulphate solution was added for further active loading of drug.

(2) Extrusion and Active Loading of Drug into Preformed Liposomes by Ammonium Sulphate Gradient

In weak base loading, 5 ml of 250 mM ammonium sulphate solution was added to hydrate the thin lipid film in the round bottom flask in a water bath of 60° C., forming multilamellar vesicles (MLVs). Next, the liposome sizes were reduced by extrusion through polycarbonate filter membranes in the corresponding size sequences of 0.2 μm (5 times), and 0.1 μm (10 times) using a bench top extruder purchased from Northern Lipids Inc, Canada. After these extrusion steps, large unilamellar vesicles (LUVs) with a size distribution of around 100±20 nm were formed.

After the LUVs were formed, the extra-liposomal ammonium sulphate solution was exchanged with an iso-osmotic salt solution of 150 mM sodium chloride (pH 5.5 adjusted) by dialysis method to set up the ammonium sulphate gradient in the liposomes. The liposome suspension was pipetted into a cellulose ester dialysis bag (100 kD MWCO, 31 mm diameter) and clipped at both ends with dialysis clips. The dialysis bag containing the liposome suspension was then suspended in a 4 litre solution of 150 mM NaCl solution (pH 5.5) at a volume ratio of 1:800 and continuously rotated for 3 hours at around 250 rpm.

Next, a known concentration of drug diluted (usually 1 mg/ml or 2 mg/ml initial loading concentration) in 150 mM NaCl solution pH 5.5 was prepared and added to the liposomes after the first dialysis step. This mixture of liposomes and drug was incubated in a 60° C. water bath with intermittent mixing every 10 minutes. Following that, the liposomal vesicles were cooled down in a 4° C. refrigerator for 1 hour to ensure stable entrapment of the drugs in the liposome.

Subsequently, the mixture of liposome was transferred into a 31 mm cellulose ester dialysis bag and clipped at both ends with dialysis clips. The dialysis bag was then suspended in 1 litre of 150 mM NaCl solution (pH 5.5) for 3 hours. At the first half hour point, 1 ml of NaCl solution from the beaker containing the dialysis bag was withdrawn and kept aside. After that, 1 ml of fresh NaCl solution was pipetted back into the beaker. At the 1^{st} hour, 2^{nd} hour and 3^{rd} hour time points, the similar step of withdrawing and replenishing 1 ml of NaCl buffer solution was carried out.

At the end of the second dialysis step, the dialysis bag was retrieved from the NaCl solution. While transferring the liposome mixture, care was taken to measure its volume. The liposome mixture was then diluted to 5 ml using NaCl solution depending on the volume of liposome mixture measured.

### 3) Measurement of Entrapped Drug Concentration

Generally, 160 μl of isopropyl alcohol (IPA) was added to a solution of 40 μl of liposomes with 1800 μl of pH 5.5 NaCl in a 2 ml micro-centrifuge tube to break the liposomes. Addition of IPA into liposomes caused the breakdown of the phospholipid bilayer, exposing the entrapped drugs within the liposomes. After centrifuging for 20 minutes at 25° C., 3 sample volumes of 100 μl from centrifuged sample was pipetted into 96 well plate and UV-spectroscopy was used to measure the entrapped concentrations. An average of the three absorbance values was corrected against absorbance values of NaCl and converted into concentration (m/m1). Next, that concentration was multiplied by 50 times according to the dilution factor for this step to derive the amount (μg) of encapsulated drug in 5 ml of liposomes. Similarly, HPLC method was also used to measure the drug concentration of liposomes

### 4) Measurement of Free Drug Concentration

For free drug concentration, the 3 sample volumes of 100 μl from the 1 ml sample taken at the 3^{rd} hour time-point were pipetted into the 96 well and UV-Vis spectroscopy was carried out. The average absorbance values of the three samples is corrected against absorbance values of NaCl and then converted into concentration (m/ml). Lastly, the concentration was multiplied by 1000 times according to the dilution factor for this step to derive the amount (μg) of free drug in 5 ml of liposomes.

### 5) In-Vitro Drug Release Study and Actual Drug Release Per Day

A dialysis method was used to evaluate the release of timolol maleate from liposomal nanocarriers. Here, the receptor medium was physically separated from the drug-loaded liposomes by a dialysis membrane. The released drug concentration was evaluated from the receptor medium over time using Ultraviolet-Visible (UV-Vis) Spectrophotometer or by HPLC.

For this study, 1 ml of drug-loaded liposomes was pipetted into a cellulose ester dialysis bag (100 kD MWCO) and clipped by dialysis clips on both ends. 40 ml PBS buffer pH 7.4 (137 mM NaCl, 2.68 mM KCl, 1.76 mM KH2PO4, 10.14 mM Na₂HPO₄) was used as a receptor medium taken in an amber bottle. The dialysis bags was suspended in the PBS buffer and placed in an incubator at a temperature of 37° C. 2 ml of dialysis medium is sampled out every day for measurement of released drug concentration and the dialysis medium was completely replenished every day.

For actual drug release, 100 μl aliquots are pipetted into 3 different wells on the 96 well plates that are used for UV measurements. Using the spectrophotometer with the wavelength set at 290 nm, the absorbance values are converted into actual drug amounts by comparison to the standard curve of timolol maleate in PBS, pH 7.4 or by using HPLC method.

Results and Data from DPPC Liposomes
Particle Size and after Extrusion

After formulating the MLVs with the thin film hydration technique, extrusion was carried out on the MLVs to form LUVs with sizes around 100±30 nm. Table 1 shows the Z-average sizes (nm) of the different batches of liposomes prepared and T₁, T₂, T₃ represent time points in months of storage at 4° C.

TABLE 1
Z-average Sizes of Liposomes

| | Liposomal | | Z-average size (nm) | | | | | |
|---|---|---|---|---|---|---|---|---|
| | Formulation | | T₁ size | Standard | | Standard | | Standard |
| Index | (mol %) | Batch | (PDI) | Deviation | T₂ (PDI) | Deviation | T₃ (PDI) | Deviation |
| P1 | DPPC/Cholesterol | 1 | 124.4 | 0.93 | 124 | 2.13 | 124.4 | 0.84 |
| | (60:40) | | (0.012) | | (0.044) | | (0.035) | |
| | | 2 | 125.7 | | 127.25 | | 125.85 | |
| | | | (0.053) | | (0.082) | | (0.065) | |
| | | 3 | 123.9 | | 123.25 | | 124.4 | |
| | | | (0.031) | | (0.047) | | (0.040) | |
| P2 | DPPC/Cholesterol | 1 | 120.5 | 1.91 | 149.5 | 18.67 | — | — |
| | (80:20) | | (0.043) | | (0.079) | | | |
| | | 2 | 123.2 | | 123.1 | | — | — |
| | | | (0.108) | | (0.109) | | | |
| P3 | DPPC (100) | 1 | 126.25 | — | 124.5 | — | — | — |
| | | | (0.087) | | (0.097) | | | |

TABLE 2
Average Batch Liposomal Size

| | Liposomal | | Z-average size (nm) | | |
|---|---|---|---|---|---|
| | Formulation | | T₁ Average | T₂ Average | T₂ Average |
| Index | (mol %) | Batch | size (nm) | size (nm) | Size (nm) |
| | DPPC/ | 1 | 124.67 | 124.83 | 124.83 |
| | Cholesterol | 2 | | | |
| P1 | (60:40) | 3 | | | |
| | DPPC/ | 1 | | | — |
| P2 | Cholesterol | 2 | 121.85 | 136.3 | — |
| | (80:20) | | | | |
| P3 | DPPC (100) | 1 | 126.25 | 124.5 | — |

Three different compositions of DPPC and cholesterol were used for this study. P1, P2 and P3 represent DPPC/Cholesterol (60:40), DPPC/Cholesterol (80:20) and DPPC (100), respectively. For each type of liposomes, at least two independent batches were formulated to test for the reproducibility of results except for the plain liposomes (i.e. DPPC (100)). All liposomes tested were loaded with timolol maleate by active loading technique described earlier.

From Table 1, it can be seen that the Z-average sizes of the P1, P2 and P3 sizes are within the range of 100±30 nm and remained reasonably unchanged for two months of storage at 4° C. According to data from Malvern Instruments Ltd and results from the Table 1, it can also be seen that the Z-average size of the liposome is independent of the composition of cholesterol in the liposome. This reproducible narrow size of liposomes was possible due to extrusion under high pressure with specified pore sizes. There were minimal changes in the average size of the liposomes with cholesterol incorporation as seen in Table 2 for most of the batches.

### Drug Loading and Release Study

The in-vitro drug release profiles of timolol maleate in PBS of pH 7.4 at of 37° C. are discussed. The main objective is to study and understand the release behavior of drugs incorporated into the lipids with and without cholesterol and evaluate the possibility of sustaining the drug release over a long period of time. To achieve optimal therapeutic level, the amount of drug released should be within the therapeutic level for it to be effective. Over-dosage of the drug can lead to negative toxic effects, while under-dosage can result in insignificant effects. Hence, sustained release within the therapeutic window is regarded an optimum mode of treatment for diseases such as glaucoma.

In this study, it is seen that liposome P1 shows a linear and sustained drug release for over 50 days with the highest composition of cholesterol as compared to P2 and P3. Addition of cholesterol to liposomes in its liquid state increases the membrane packing parameter thereby reducing membrane fluidity. Due to this, the phospholipid will retain the entrapped drug better and give a controlled release over a long period of time.

The amount of entrapped drug was calculated after breaking down the liposomes and shown in Table 3, which shows the amount of encapsulated drug per 1 ml for liposomes P1, P2 and P3.

TABLE 3
Encapsulated Drug Amount in Liposomes

| | | | | Percentage | Average ± |
|---|---|---|---|---|---|
| | Liposomal | | Encapsulated | Encapsulation | Standard |
| In- | Formulation | | Amount | Efficiency | Deviation |
| dex | (mol %) | Batch | (μg/ml) | (%) | (%) |
| P1 | DPPC/ | 1 | 802.2 | 80.2 | 74.37 ± 5.08 |
| | Cholesterol | 2 | 708.9 | 70.9 | |
| | (60:40) | 3 | 720.1 | 72.0 | |
| P2 | DPPC/ | 1 | 454.5 | 45.5 | 46.30 ± 1.13 |
| | Cholesterol | 2 | 470.5 | 47.1 | |
| | (80:20) | | | | |
| P3 | DPPC (100) | 1 | 713.2 | 71.3 | — |

It can be seen from Table 3 that the average amount of drugs encapsulated per 1 ml for the different batches of liposomes ranges between 40-80% and high encapsulation efficiencies were obtained by active loading technique.

### Drug Release Studies

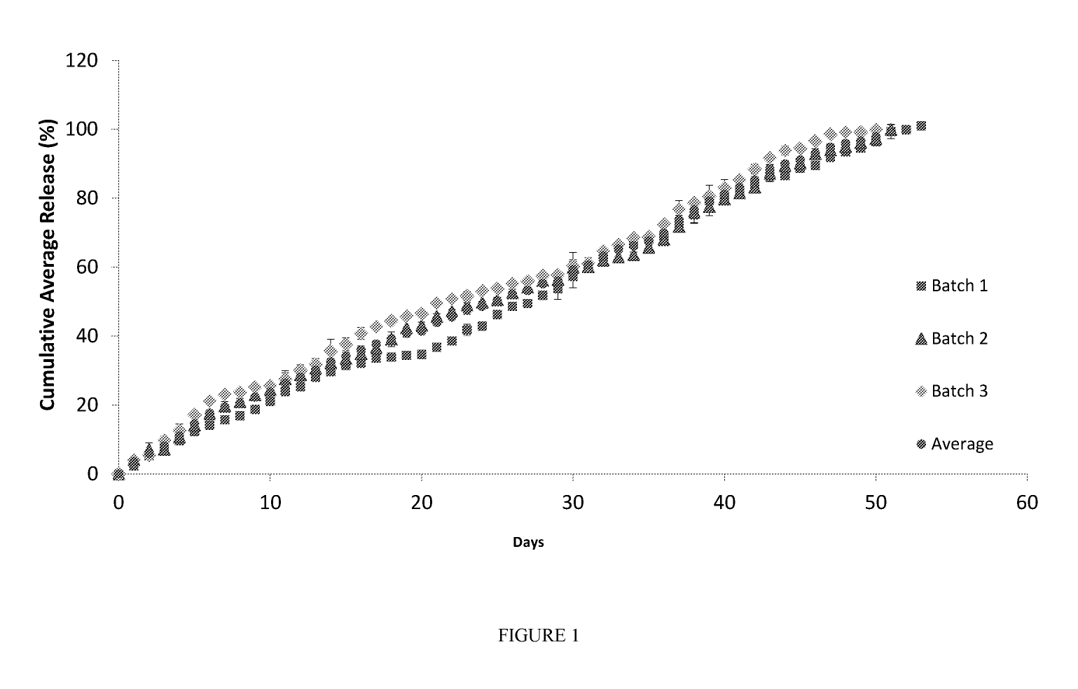

FIG. 1 shows the percentage cumulative average release versus days of liposome P1. From FIG. 1, it can be seen that all three batches of P1 showed slow and sustained release of drug up to 50 days and reproducible for the three independent batches prepared. The average drug release per day remains relatively constant and burst is minimal.

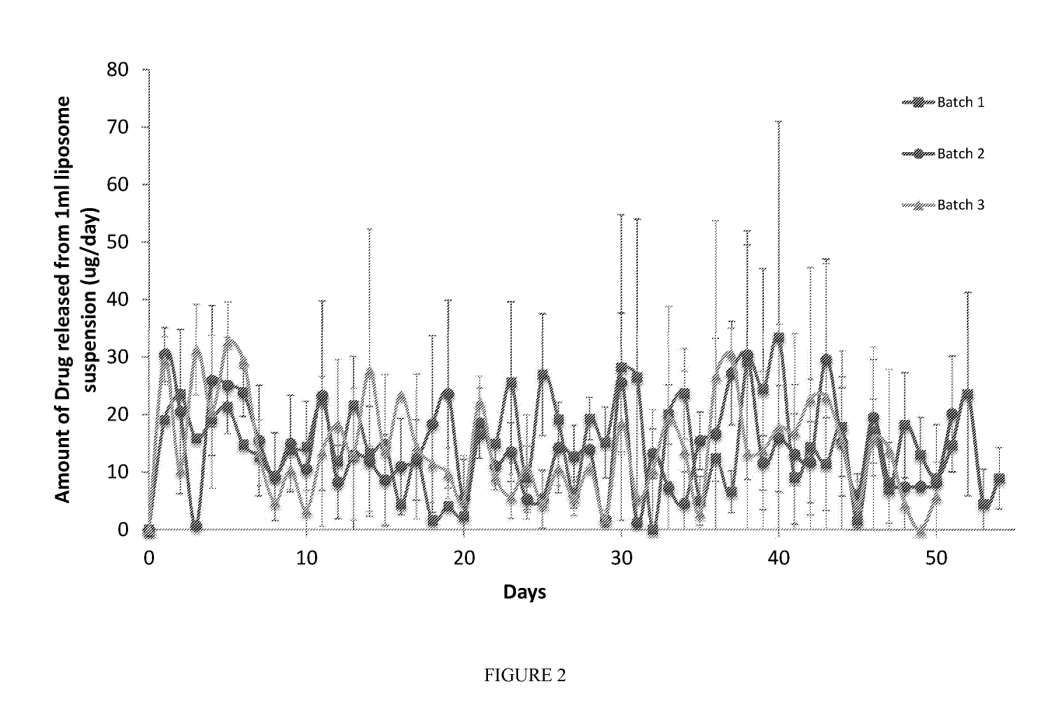

FIG. 2 shows the amount of drug released from 1 ml of liposome suspension of P1. As shown in FIG. 2, the release is controlled within the range of 0 μg/ml to 35 μg/ml and lasted for around 50 days.

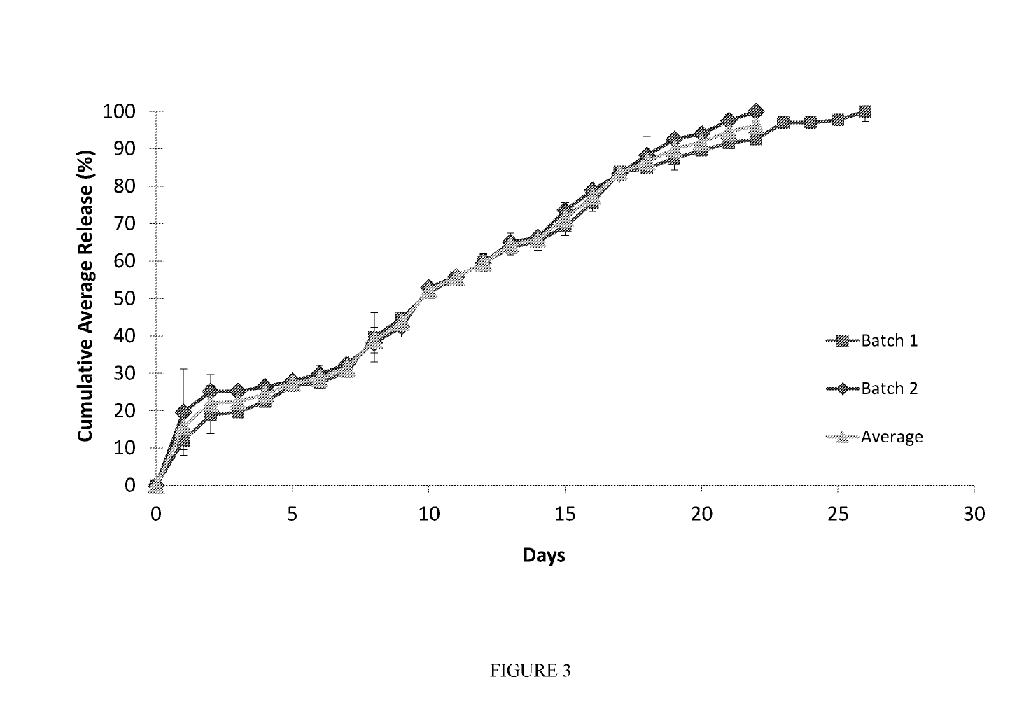

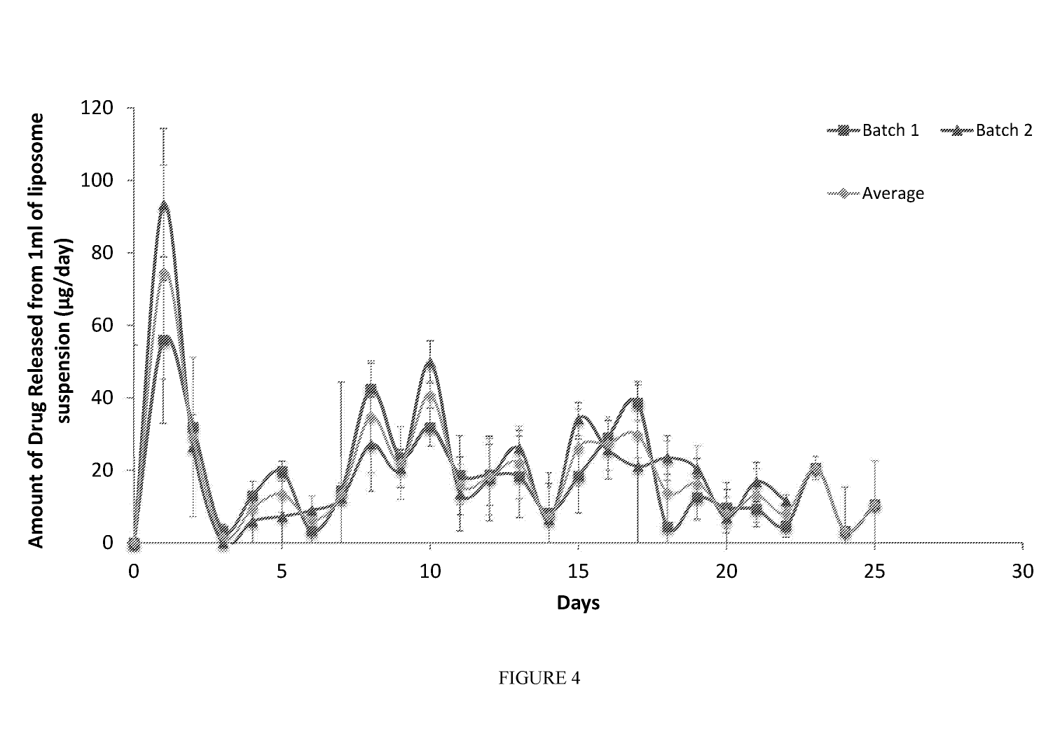

Next, the drug release behavior of liposome P2 is studied. FIG. 3 shows the percentage cumulative average release versus days of P2. FIG. 4 shows the amount of drug released from 1 ml of liposome suspension of P2. From FIG. 3, it is observed a slow and sustained release of the drug up to 25 days for liposome P2. It is, however, evident from the FIG. 4 that there is a relatively larger burst in drug release on the first day. As seen from FIG. 4, liposome P2 batch 2 had a sudden release of 93.3 μg of timolol maleate within one day. The release was sustained for at least three weeks with 20 mol % of cholesterol included in the liposomes.

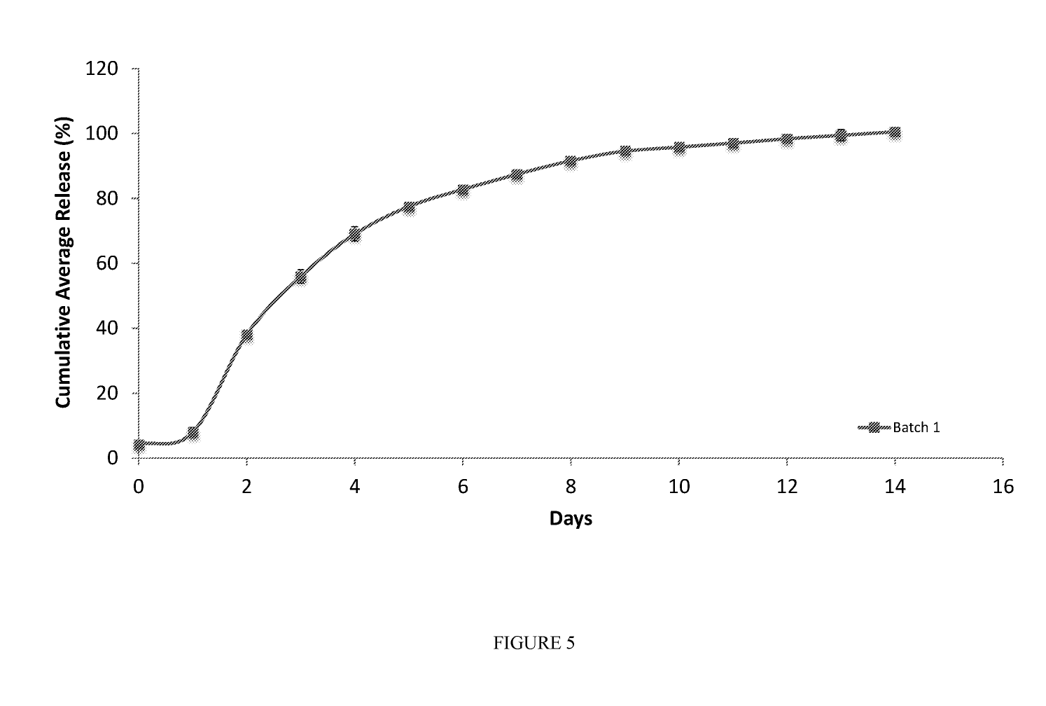

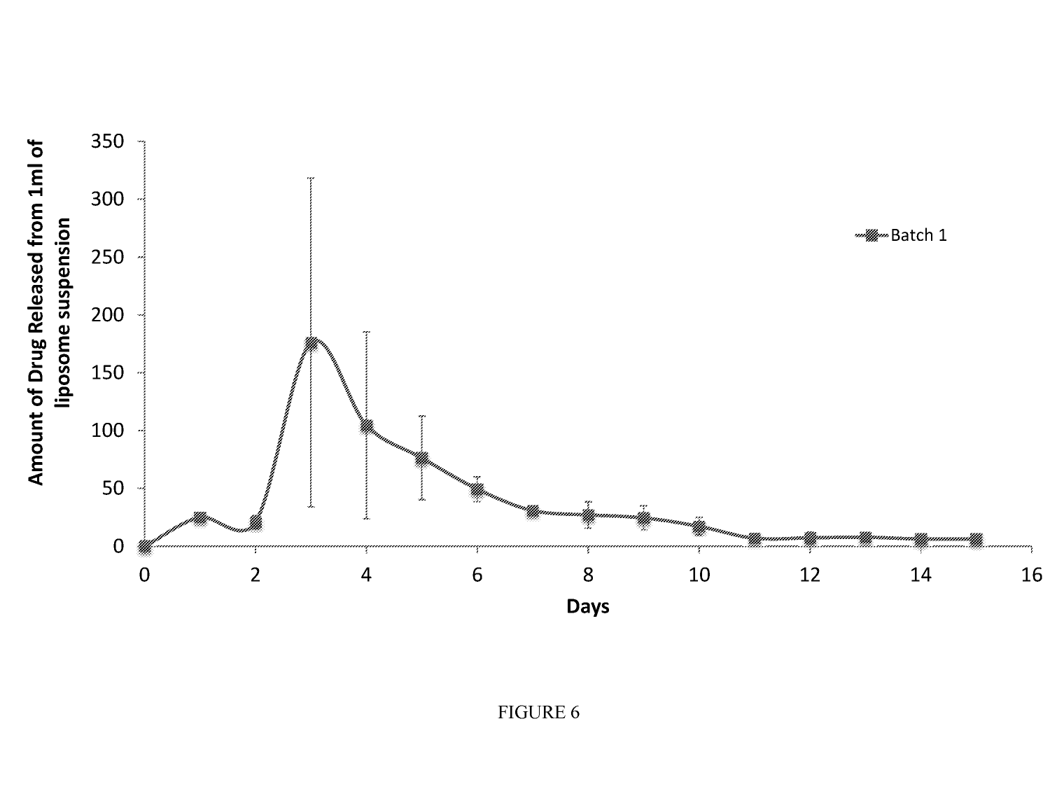

Next, we will discuss on the release behavior from plain liposomes P3. FIG. 5 shows the percentage cumulative average release versus days of liposome P3. FIG. 6 shows the amount of drug released from 1 ml of liposome suspension of P3. As seen from FIG. 5, drug release for liposome P3 ended within 15 days with a relatively higher burst as shown in FIG. 6. The drug release started off with larger amount of release for the first few days. Nearing to day 10 onwards, there was negligible amount of drug release per day as observable from FIG. 6, suggesting that release could not sustained beyond two weeks.

### Comparison Between Plain DDPC Liposomes, DPPC/Cholesterol (80:20) Liposomes and DPPC/Cholesterol (60:40) Liposomes

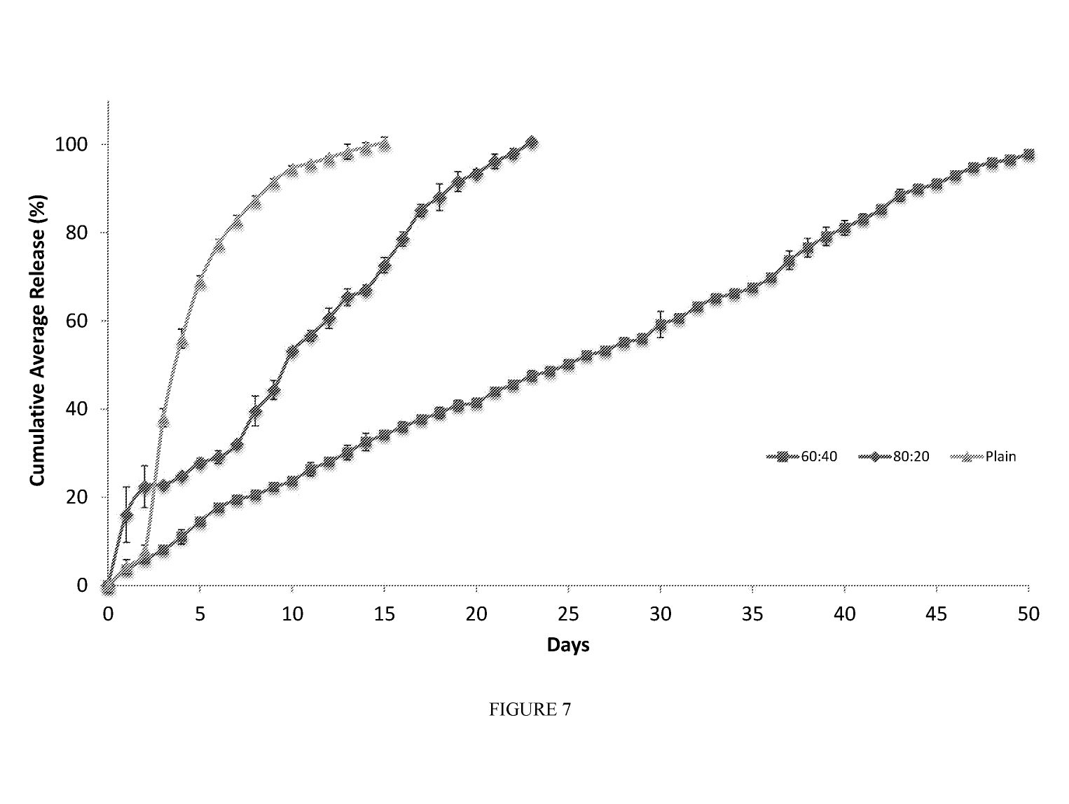

FIG. 7 shows a comparison of cumulative average release of timolol maleate from plain, DPPC/Cholesterol (80:20 mol %), and DPPC/Cholesterol (60:40 mol %) liposomes according to the examples. From FIG. 7, drug release behaviors of liposome P1, P2 and P3 suggest that liposomes with the highest cholesterol composition, P1, will have the most ideal controlled and sustained drug release profile as compared to the other liposomes as incorporation of cholesterol in higher concentrations (usually ≧20 mol %) improves rigidity of the bilayer thus slowing the diffusion rate of release from the core to the external medium. A concentration dependent, slow and sustained release was observed with increase in cholesterol amounts. Liposome P1 has the longest and most sustained drug release, for around 50 days, as compared to liposomes P2 and P3. This is due to the higher composition of cholesterol in liposome P1 (40 mol %). In general, cholesterol helps to increase the hydrophobicity of the phospholipid bilayer membrane and condensed the membrane which was shown to be dependent on cholesterol concentration. In addition, cholesterol content in liposome contributes to rigidity of the membrane structure by controlling permeability and increasing plasma stability.

Batches of liposomes P2 reached 100% drug release at a maximum of 25 days, while the batch of liposome P3 completed its drug release in 15 days. This may be due to the lower concentration of cholesterol in the liposomes, resulting in lower membrane rigidity and a less sustained drug release. In addition, liposome P2 displayed a large burst of timolol maleate release on the first day, which is not desired. Similarly in liposome P3, there is a burst of timolol maleate release on day 3, which accounts for near to 30% of the entrapped drug within the liposome. Therefore, it can be understood that with lower cholesterol contents in the composition, liposomes P2 and P3 are not as stable and capable of sustained drug release as compared to liposome P1, with the highest concentration of cholesterol.

### Timolol Maleate Loaded in Negatively and Positively Charged Liposomes

### Negatively Charged Liposomes

### (DPPC/DPPG, 80:20 mol %),

DPPG—1,2-dipalmitoyl-sn-glycero-3-phospho-(1′-rac-glycerol) (sodium salt)
DPPC—1,2-dipalmitoyl-sn-glycero-3-phosphocholine
Initial lipid concentration: 18 mM
Loading of drug: Active loading by ammonium sulphate gradient

### Average Size: 102.3 nm

TABLE 4
Entrapped drug concentration for negatively charged liposomes

| | | | Encapsula- | Initial |
|---|---|---|---|---|
| | | Entrapped drug | tion effi- | total drug |
| S. | Description | concentration | ciency | concentration |
| no. | (in mol %) | (mg/ml) | (%) | (mg/ml) |
| 1) | DFPC (80), | 0.664 ± 0.008 | 61.06 ± 0.3 | 1 |
| | DPPG (20) | | | |

### Positively Charged Liposomes

### (DPPC/DOTAP, 80:20 mol %),

DPPG—1,2-dipalmitoyl-sn-glycero-3-phospho-(1′-rac-glycerol) (sodium salt)
DOTAP—1,2-dioleoyl-3-trimethylammonium-propane
Initial lipid concentration: 18 mM
Loading of drug: Active loading by ammonium sulphate gradient

### Average Size: 108.8 nm

TABLE 5
Entrapped drug concentration for positively charged liposomes

| | | | Encapsula- | Initial |
|---|---|---|---|---|
| | | Entrapped drug | tion effi- | total drug |
| S. | Description | concentration | ciency | concentration |
| no. | (in mol %) | (mg/ml) | (%) | (mg/ml) |
| 1) | DPPC (80), | 0.515 | 51.5 | 1 |
| | DOTAP (20) | | | |

### Preparation of Drug-Free Preformed Liposomes

Firstly, lipid was placed in a vacuum desiccator for 1 hour to remove any residual moisture before weighing. Each batch of liposome was prepared in 5 ml per batch, with an initial lipid concentration of 18 millimolar (mM). Plain DPPC, DPPC/DPPG (80:20 mol %), DPPC/DOTAP (80:20 mol %) were prepared. Lipids in fixed molar ratios were measured and taken in a round bottom flask and dissolved in an organic phase mixture that contained methanol and chloroform in a ratio of 1:2. Subsequently, the flask was rotated in the rotary operator and operated under reduced pressure for 1 hour maintained in a water bath temperature of 40° C. to remove the organic phase, ultimately leaving behind a thin film of lipids covering the bottom of the flask. To the thin lipid film, 5 ml of ammonium sulphate solution was added for further active loading of drug.

The rest of the processes remain the same as those described earlier for the preparation of timolol maleate liposomes.

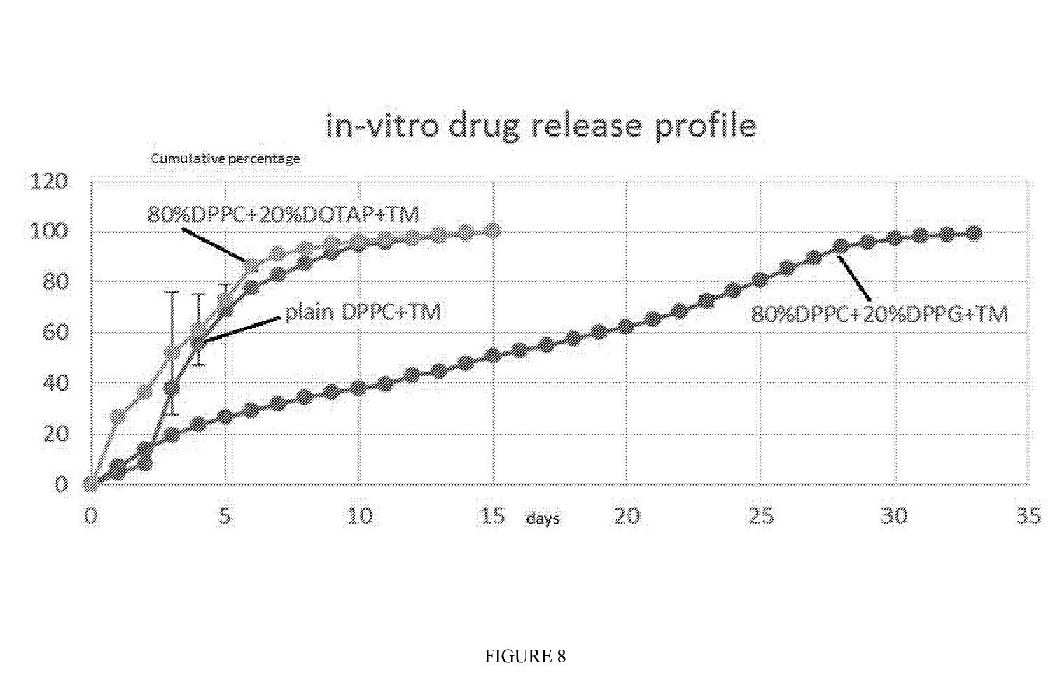

FIG. 8 shows the in vitro drug release profiles from plain, DPPC/DPPG (80:20 mol %), and DPPC/DOTAP (80:20 mol %) liposomes.

Results and Data from DPPC and Sphingomyelin Liposomes

TABLE 6
Entrapped drug concentration

| | | | Encapsula- | Initial |
|---|---|---|---|---|
| | | Entrapped drug | tion effi- | total drug |
| S. | Description | concentration | ciency | concentration |
| no. | (in mol %) | (mg/ml) | (%) | (mg/ml) |
| 1) | DP(80)/Chol(20) (a) | 0.67 | 33.5 | 2 |
| 2) | DP(70)/Chol(30) (b) | 1.22 | 61 | 2 |
| 3) | DP(65)/Chol(35) (c) | 1.07 | 53.5 | 2 |
| 4) | DP(63)/Chol(37) (d) | 0.96 | 48 | 2 |
| 5) | DP(60)/Chol(40) (e) | 1.32 | 66 | 2 |

DP—DPPC; Chol—Cholesterol. The number in the brackets denotes mol %

A high entrapped drug concentration (up to 1.5 mg/ml) using an ammonium sulphate gradient (Table 6) for an initial total drug concentration of 2 mg/ml has been achieved.

High encapsulation efficiency was obtained for liposomes with 40 mol % cholesterol for an initial total drug concentration of 1 mg/ml.

TABLE 7
Entrapped drug concentration

| | | | | Initial |
|---|---|---|---|---|
| | | Entrapped drug | Encapsulation | total drug |
| S. | Description | concentration | efficiency | concentration |
| no. | (in mol %) | (mg/ml) | (%) | (mg/ml) |
| 1) | DP(60)/Chol(40) | 0.854 | 85.4 | 1 |
| 2) | SP(60)/Chol(40) | 0.63 | 63 | 1 |

DP—DPPC; Chol—Cholesterol; SP—sphingomyelin. The number in brackets denotes mol %.

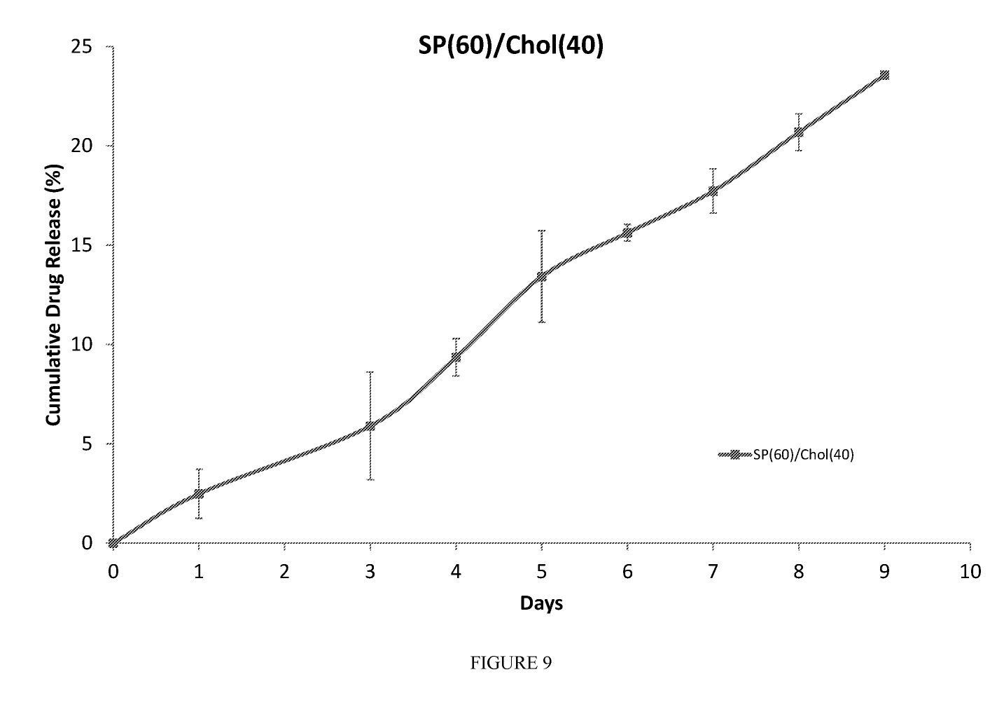

FIG. 9 shows the cumulative release of timolol maleate from sphingomyelin liposomes containing 40 mol % cholesterol. As shown in FIG. 9, it is observed an almost linear release profile from sphingomyelin liposomes that contain (40 mol %) cholesterol. The release was slow and sustained with ˜25% of the drug being released by the end of ten days. These studies confirm the possibility of sustained efficacy of action (IOP lowering) when tested in vivo, based on previous experience with latanoprost delivery using liposomal nanocarriers.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

By “about” in relation to a given numerical value, such as for temperature and period of time, it is meant to include numerical values within 10% of the specified value.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

1. Liposomal formulation for ocular drug delivery, comprising:
liposomes each comprising a core surrounded by one or more lipid bilayers, and timolol maleate comprised in the core of each liposome,
(a) wherein one or more of the lipid bilayers are comprised of a mixture of 50-80 mol % of a lipid and 20-50 mol % of a steroid alcohol, wherein the lipid is comprised of glyceride, phosphatidylcholine, and/or sphingolipid; or
(b) wherein the one or more lipid bilayers are comprised of a mixture of 50-80 mol % of a neutral lipid and 20-50 mol % of a charged lipid, wherein the neutral lipid and the charged lipid are comprised of glyceride, phosphatidylcholine, and/or sphingolipid.

2. Liposomal formulation according to claim 1, wherein the one or more lipid bilayers are comprised of 30-50 mol % of the steroid alcohol, preferably 30 mol %, 35 mol %, 37 mol %, 40 mol %, 45 mol %, or 50 mol %.

3. Liposomal formulation according to claim 1 or 2, wherein the steroid alcohol comprises phytosterol, zoosterol, or a mixture thereof.

4. Liposomal formulation according to claim 3, wherein the steroid alcohol has a general formula 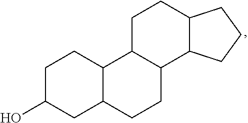
wherein any of the carbon atom is optionally substituted, preferably with a C1-C10 alkyl.

5. Liposomal formulation according to claim 4, wherein the steroid alcohol has the following structure

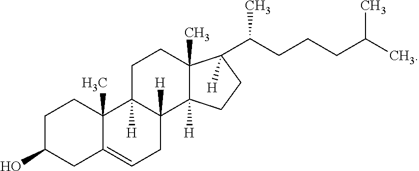

6. Liposomal formulation according to any one of the preceding claims, wherein the lipid and/or the neutral lipid comprises one of phosphatidylcholine and sphingolipids.

7. Liposomal formulation according to claim 6, wherein the phosphatidylcholine is selected from the group consisting of 1,2-dioleoyl-sn-glycero-3-phosphocholine (DOPC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1-palmitoyl-2-oleoyl-sn-glycero-3-phosphocholine (POPC), egg phosphatidylcholine (egg PC), soy phosphatidylcholine (soy PC), hydrogenated phosphatidylcholine (HPC), and a mixture thereof.

8. Liposomal formulation according to claim 7, wherein the phosphatidylcholine comprises DPPC having the following structure

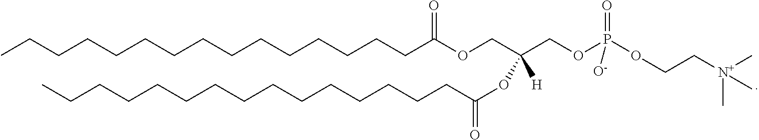 [SMILES: CCCCCCCCCCCCCCCC(=O)OC[C@H](COP(=O)([O-])OCC[N+](C)(C)C)OC(=O)CCCCCCCCCCCCCCC]

9. Liposomal formulation according to claim 6, wherein the sphingolipid comprises shingomyelin from egg having the following structure

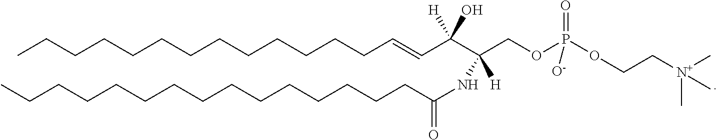 [SMILES: CCCCCCCCCCCCC/C=C/[C@@H](O)[C@H](COP(=O)([O-])OCC[N+](C)(C)C)NC(=O)CCCCCCCCCCCCCCC]

10. Liposomal formulation according to any one of the preceding claims, wherein each liposome comprises 0.67-1.5 mg/ml of entrapped timolol maleate concentration based on an initial timolol maleate concentration of 2 mg/ml.

11. Liposomal formulation according to any one of claims 1-9, wherein each liposome comprises 0.63-0.85 mg/ml of entrapped timolol maleate concentration based on an initial timolol maleate concentration of 1 mg/ml.

12. Liposomal formulation according to any one of the preceding claims, wherein timolol maleate is comprised in the core and another ocular drug is comprised in the lipid bilayer of each liposome.

13. Liposomal formulation according to any one of the preceding claims, further comprising:
additional liposomes each comprising a core surrounded by one or more lipid bilayers; and
another ocular drug different from timolol maleate comprised in the one or more lipid bilayers of each additional liposome.

14. Liposomal formulation according to claim 13, wherein the another ocular drug is selected from the group consisting of latanoprost, bimatoprost, travoprost, carboprosttrometamol, gemeprost, sulprostone, dinoprostone (PGE2), alprostadil (PGE1), beroprost, iloprost, epoprostenol, treprostinil, misoprostol, enoprostil, omoprostil, limaprost. unoprostone isopropyl, arthrotec, and a mixture thereof.

15. Liposomal formulation according to claim 14, wherein the another ocular drug is latanoprost.

16. Liposomal formulation according to any one of the preceding claims, wherein the liposomal formulation comprises a timolol maleate to lipid mole ratio of about 0.01 to about 0.30, preferably 0.01, 0.05, 0.10, 0.15, 0.20, 0.25, or 0.30.

17. Liposomal formulation according to claim 1, wherein the lipid bilayer is comprised of a neutral lipid and a negatively charged lipid.

18. Liposomal formulation according to claim 16, wherein the neutral lipid comprises 1,2-dipalmitoyl-sn-glycero-3-phosphocholine (DPPC), 1,2-dioleoyl-sn-glycero-3-phosphocholines (DOPC), 1,2-Dilauroyl-sn-glycero-3-phosphocholines (DLPC), 1,2-Dimyristoyl-sn-glycero-3-phosphocholines (DMPC), 1,2-Distearoyl-sn-glycero-3-phosphocholines (DSPC), L-α-phosphatidylcholine or 95% Egg phosphatidylcholines (eggPC 95%), 1-palmitoyl-2-oleoylphosphatidylcholine (POPC), L-α-phosphatidylcholine, hydrogenated (Soy) or mixtures thereof.

19. Liposomal formulation according to claim 17 or 18, wherein the negatively charged lipid comprises 1,2-dipalmitoyl-sn-glycero-3-phospho-(1′-rac-glycerol) (sodium salt) (DPPG), (L-α-phosphatidylglycerol (Egg, Chicken) (sodium salt) (EggPG), L-α-phosphatidylglycerol (Soy) (sodium salt) (Soy PG), 1,2-dimyristoyl-sn-glycero-3-phospho-(1′-rac-glycerol) (sodium salt) (DMPG), 1,2-dilauroyl-sn-glycero-3-phospho-(1′-rac-glycerol) (sodium salt) (DLPG), 1,2-distearoyl-sn-glycero-3-phospho-(1′-rac-glycerol) (sodium salt), L-α-phosphatidic acid (Egg, Chicken) (sodium salt) (EggPA), L-α-phosphatidic acid (Soy) (sodium salt) (SoyPA), 1,2-dilauroyl-sn-glycero-3-phosphate (sodium salt) (DLPA), 1,2-dimyristoyl-sn-glycero-3-phosphate (sodium salt) (DMPA), 1,2-dipalmitoyl-sn-glycero-3-phosphate (sodium salt) (DPPA), 1,2-distearoyl-sn-glycero-3-phosphate (sodium salt) (DSPA) and mixtures thereof.

20. Liposomal formulation according to claim 19, wherein the one or more lipid bilayers comprise 80 mol % of DPPC and 20 mol % DPPG.

21. Liposomal formulation according to claim 1, wherein the one or more lipid bilayers are comprised of a neutral lipid and a positively charged lipid.

22. Liposomal formulation according to claim 21, wherein the neutral lipid comprises 1,2-dipalmitoyl-sn-glycero-3-phosphocholine (DPPC), (1,2-dioleoyl-sn-glycero-3-phosphocholines (DOPC), 1,2-Dilauroyl-sn-glycero-3-phosphocholines (DLPC), 1,2-Dimyristoyl-sn-glycero-3-phosphocholines (DMPC), 1,2-Distearoyl-sn-glycero-3-phosphocholines (DSPC), L-α-phosphatidylcholine or 95% Egg phosphatidylcholines (eggPC 95%), 1-palmitoyl-2-oleoylphosphatidylcholine (POPC), L-α-phosphatidylcholine, hydrogenated (Soy) (HSPC) and mixtures thereof.

23. Liposomal formulation according to claim 21 or 22, wherein the positively charged lipid comprises 1,2-dioleoyl-3-trimethylammonium-propane (DOTAP), (1,2-dioleoyl-3-trimethylammonium-propane (chloride salt) (DOTAP), 1,2-dimyristoyl-3-trimethylammonium-propane (chloride salt) (DMTAP), 1,2-dipalmitoyl-3-trimethylammonium-propane (chloride salt) (DPTAP), 1,2-stearoyl-3-trimethylammonium-propane (chloride salt) (DSTAP), Dimethyldioctadecylammonium (Bromide S alt) (DDAB), 1,2-di-0-octadecenyl-3-trimethylammonium propane (chloride salt) (DOTMA) and mixtures thereof.

24. Liposomal formulation according to claim 23, wherein the one or more lipid bilayers comprise 80 mol % of DPPC and 20 mol % DOTAP.

25. Pharmaceutical composition comprising a pharmaceutically acceptable carrier and a liposomal formulation according to any one of claims 1-24.

26. Pharmaceutical composition according to claim 25, wherein the pharmaceutical composition is in a form of an ophthalmic solution.

27. Pharmaceutical composition according to claim 26, wherein the pharmaceutical composition is for use in ocular drug delivery in an injection solution or a viscous aqueous vehicle.

28. Pharmaceutical composition according to claim 27, wherein the viscous aqueous vehicle comprises an aqueous solution of polysaccharides.

29. Pharmaceutical composition according to claim 28, wherein the polysaccharide is hyaluronic acid.

30. Use of a liposomal formulation according to any one of claims 1-24 or a pharmaceutical composition according to any one of claims 25-29 in the treatment or prevention of glaucoma or ocular hypertension, comprising administering the liposomal formulation or the pharmaceutical composition to a subject in need thereof.

31. Method of providing a sustained release of timolol maleate of minimum 10 days, comprising administering a liposomal formulation accordingly to any one of claims 1-24 or a pharmaceutical composition according to any one of claims 25-29 by subconjunctival injection.

32. Method of preparing a liposomal formulation comprising liposomes each comprising a core surrounded by one or more lipid bilayers, and timolol maleate comprised in the core of each liposome, wherein the one or more lipid bilayers are comprised of a mixture of 50-80 mol % of a lipid and 20-50 mol % of a steroid alcohol, wherein the lipid is comprised of glyceride, phosphatidylcholine, and/or sphingolipid, the method comprising:
mixing the lipid with the steroid alcohol in an organic phase;
evaporating the organic phase mixture to obtain a thin film of the lipid and steroid alcohol;
hydrating the thin film with ammonium sulfate solution to form vesicles;
suspending the vesicles in a salt solution;
adding a drug solution to the suspension; and
incubating the suspension containing the vesicles and drug solution.

33. Method of preparing a liposomal formulation comprising liposomes each comprising a core surrounded by one or more lipid bilayers, and timolol maleate comprised in the core of each liposome, wherein the one or more lipid bilayers are comprised of a mixture of 50-80 mol % of a neutral lipid and 20-50 mol % of a charged lipid, wherein the neutral lipid and the charged lipid are comprised of glyceride, phosphatidylcholine, and/or sphingolipid, the method comprising:
mixing the neutral lipid and the charged lipid in an organic phase;
evaporating the organic phase mixture to obtain a thin film;
hydrating the thin film with ammonium sulfate solution to form vesicles;
suspending the vesicles in a salt solution;
adding a drug solution to the suspension; and
incubating the suspension containing the vesicles and drug solution.
